(19) United States
(12) Patent Application Publication
(10) Pub. No.: US 2025/0179520 A1
(43) Pub. Date: Jun. 5, 2025

(54) RECOMBINANT AAV VECTORS AND USES THEREOF

(71) Applicant: University of Massachusetts, Boston, MA (US)

(72) Inventors: Guangping Gao, Worcester, MA (US); Jun Xie, Worcester, MA (US); Hyejin Oh, Worcester, MA (US)

(73) Assignee: University of Massachusetts, Boston, MA (US)

(21) Appl. No.: 18/844,488

(22) PCT Filed: Mar. 8, 2023
(86) PCT No.: PCT/US2023/063951

§ 371 Date: Sep. 6, 2024

Related U.S. Application Data
(60) Provisional application No. 63/318,107, filed on Mar. 9, 2022.

(51) Int. Cl. C12N 15/86; C12N 15/113

(52) U.S. Cl. CPC C12N 15/86; C12N 15/1138; C12N 2310/531; C12N 2750/14122; C12N 2750/14143; C12N 2750/14152

(57) ABSTRACT

Aspects of the disclosure relate to compositions, such as rAAV vectors, comprising one or more short hairpin nucleic acids positioned outside of the inverted terminal repeats (ITRs) of the rAAV vector (referred to in some embodiments as “stopper DNA”). The disclosure is based, in part, on rAAV vectors comprising stopper DNA, which have improved packaging and/or immunogenicity relative to previously described rAAV vectors. In some embodiments, the disclosure relates to methods of delivering a transgene to a subject comprising administering the rAAV vectors.

### RELATED APPLICATIONS

This application is a national stage filing under 35 U.S.C. § 371 of international PCT application PCT/US2023/063951, filed Mar. 8, 2023, which claims priority under 35 U.S.C. § 119(e) to U.S. provisional patent application, U.S. Ser. No. 63/318,107, filed Mar. 9, 2022, the entire contents of each of which are incorporated by reference herein.

### REFERENCE TO AN ELECTRONIC SEQUENCE LISTING

The contents of the electronic sequence listing (U012070153US02-SEQ-KZM.xml; Size: 1,802 bytes; and Date of Creation: Aug. 20, 2024) is herein incorporated by reference in its entirety.

### BACKGROUND

Adeno-associated viral (AAV) vectors have emerged as one of the most advanced gene transfer systems in clinical gene therapy. However, AAV vector manufacturing continues to have many challenges, for example production of high quality and quantity vectors for therapeutical administration due to vector impurities. One such impurity, non-vector DNA, is derived from replication-competent AAVs, and/or pseudo-wild type AAVs, host genomes, and can also be reverse packaged prokaryotic DNA sequences from the vector plasmid backbones. Reverse packaged prokaryotic DNA, including antibiotic resistance genes, and other plasmid backbone nucleotides, should be avoided during gene transfer for clinical safety concerns and the high risk of evoking potential immune responses and other untoward effects in recipient patients.

### SUMMARY

Aspects of the disclosure relate to compositions, such as rAAV vectors, comprising one or more short hairpin nucleic acids positioned outside of the inverted terminal repeats (ITRs) of the rAAV vector. In some embodiments, the short hairpin nucleic acids are referred to as “stopper DNA”. The disclosure is based, in part, on rAAV vectors comprising stopper DNA (e.g., short hairpin DNA (shDNA) stopper sequences), which have improved packaging and/or immunogenicity relative to previously described rAAV vectors. In some embodiments, the disclosure relates to methods of delivering a transgene to a subject comprising administering the rAAV vectors.

In some aspects, the present disclosure provides an isolated nucleic acid comprising a recombinant adeno-associated virus (rAAV) vector, and (i) a 5′ hairpin-forming nucleic acid sequence located 5′ to a 5′ AAV inverted terminal repeat (ITR) of the rAAV vector; (ii) a 3′ hairpin-forming nucleic acid sequence located 3′ to a 3′ AAV inverted terminal repeat (ITR) of the rAAV vector; or (iii) a 5′ hairpin-forming nucleic acid sequence located 5′ to a 5′ AAV inverted terminal repeat (ITR) and a 3′ hairpin-forming nucleic acid sequence located 3′ to a 3′ AAV inverted terminal repeat (ITR) of the rAAV vector.

In some embodiments, each hairpin-forming nucleic acid sequence independently encodes a short hairpin RNA (shRNA), a microRNA (miRNA), or an artificial miRNA (amiRNA).

In some embodiments, each of the hairpin-forming nucleic acid sequences ranges from about 5 nucleotides in length to about 150 nucleotides in length (e.g., 5, 6, 7, 8, 9, 10, 11, 12, 13, 14, 15, 16, 17, 18, 19, 20, 21, 22, 23, 24, 25, 50, 75, 100, 125, or 150 nucleotides in length).

In some embodiments, the 5′ hairpin-forming nucleic acid sequence further comprises a toll-like receptor 9 (TLR9)-inhibitory sequence. In some embodiments, the 3′ hairpin-forming nucleic acid sequence further comprises a toll-like receptor 9 (TLR9)-inhibitory sequence. In some embodiments, the 5′ hairpin-forming nucleic acid sequence and the 3′ hairpin-forming nucleic acid further comprise a toll-like receptor 9 (TLR9)-inhibitory sequence.

In some embodiments, the AAV vector is a single-stranded rAAV (ssAAV) vector. In some embodiments, the AAV vector is a self-complementary rAAV (scAAV) vector.

In some embodiments, the 5′ ITR and/or 3′ ITR is an AAV1, AAV2, AAV3, AAV4, AAV5, AAV6, AAV7, AAV8, or AAV9 ITR.

In some embodiments, the rAAV vector further comprises a transgene.

In some embodiments, the present disclosure provides a recombinant adeno-associated (rAAV) virus comprising: (i) the isolated nucleic acid as described herein; and (ii) an adeno-associated virus (AAV) capsid protein.

In some embodiments, the AAV capsid protein is of a serotype selected from AAV1, AAV2, AAV3, AAV4, AAV5, AAV6, AAV7, AAV8, AAV9, AAV.rh32.33, and a variant of any of the foregoing.

In some aspects, the present disclosure also provides a cell comprising the isolated nucleic acid described herein, or the rAAV described herein.

In some aspects, the present disclosure also provides a pharmaceutical composition comprising the isolated nucleic acid described herein, the rAAV described herein, or the cell described herein. In some embodiments, the pharmaceutical composition further comprising a pharmaceutically acceptable carrier.

In some aspects, the present disclosure also provides a method of reducing reverse packaging of a recombinant adeno-associated virus (rAAV) during rAAV production, the method comprising delivering to host cells: (i) the isolated nucleic acid described herein; (ii) a first vector encoding one or more helper gene; and (iii) a second vector encoding a AAV replication gene and a AAV capsid gene.

In some aspects, the present disclosure provides a method of reducing reverse packaging of a recombinant adeno-associated virus (rAAV) during rAAV production, the method comprising delivering to host cells: (i) the isolated nucleic acid comprising a recombinant adeno-associated virus (rAAV) vector and (a) a 5′ hairpin-forming nucleic acid sequence located 5′ to a 5′ AAV inverted terminal repeat (ITR); (b) a 3′ hairpin-forming nucleic acid sequence located 3′ to a 3′ AAV inverted terminal repeat (ITR); or (c) a 5′ hairpin-forming nucleic acid sequence located 5′ to a 5′ AAV inverted terminal repeat (ITR) and a 3′ hairpin-forming nucleic acid sequence located 3′ to a 3′ AAV inverted terminal repeat (ITR); (ii) a first vector encoding one or more helper gene; and (iii) a second vector encoding a AAV replication gene and a AAV capsid gene.

In some embodiments, the host cells are human cells or insect cells. In some embodiments, the human cells are HEK293 cells or Hela cells.

### BRIEF DESCRIPTION OF DRAWINGS

FIG. 1 shows a schematic depicting one embodiment of rAAV vectors comprising shDNA “stoppers” positioned outside of the rAAV vector 5′ and 3′ ITRs. The shDNA sequence used was 5′-TTAGGGTTAGGGTTAGGGTTAGGGTTCAAGAGAccctaaccctaaccctaaccctaa-3′ (SEQ ID NO: 1).

FIG. 2 shows genome populations measured by ddPCR targeting different vector regions in purified vectors; V5495: 5′shDNA-scAAVrh32.33.CB6-EGFP; V5496: scAAVrh32.33.CB6-EGFP-shDNA3′; V5497:5′shDNA-scAAVrh32.33.CB6-EGFP-shDNA3′; V5498:scAAVrh32.33.CB6-EGFP.

FIGS. 3A-3D show representative data for single-molecule real-time (SMRT) sequencing of self-complementary AAVrh32.33 vectors comprising shDNA stopper-linked ITRs. FIG. 3A depicts a schematic of the composition of the base scAAVrh32.33 vector lacking shDNA elements. FIG. 3B depicts a schematic of the composition of the scAAVrh32.33 stopper vector comprising shDNA elements on the 5′ and 3′ ends of the ITRs. Below both schematics shown in FIGS. 3A and 3B are visual representations of the SMRT sequencing results corresponding to the different regions of the vectors. FIG. 3C shows a graph of the quantity of non-vector genome packaging detected in the 5′ region of the base scAA Vrh32.33 vector (5′ sc) versus in the scAAVrh32.33 stopper vector containing 5′ and 3′ shDNA stopper sequences (5′ sc stopper). FIG. 3D shows a graph of the quantity of non-vector genome packaging detected in the 3′ region of the base scAAVrh32.33 vector (3′ sc) vector versus in the scAAVrh32.33 stopper vector containing 5′ and 3′ shDNA stopper sequences (3′ sc stopper).

### DETAILED DESCRIPTION

Aspects of the disclosure relate to compositions and methods for reducing reverse packaging (e.g., packaging of prokaryotic DNA, such as an antibiotic resistance gene and other plasmid backbone sequences, from vector plasmid backbones) into rAAV particles (e.g., during rAAV production). Aspects of the disclosure relate to compositions, such as rAAV vectors, comprising one or more hairpin-forming nucleic acids positioned outside of the inverted terminal repeats (ITRs) of the rAAV vector. In some embodiments, inclusion of the hairpin-forming nucleic acids outside one or both ITRs have improved packaging and/or immunogenicity relative to previously described rAAV vectors. In some embodiments, the disclosure provides method of producing rAAV particles (e.g., rAAV particles). In some embodiments, the disclosure relates to methods of delivering a transgene to a subject comprising administering the rAAV vectors.

### Isolated Nucleic Acids

In some aspects, the disclosure provides an isolated nucleic acid comprising a recombinant adeno-associated virus (rAAV) vector, and (i) a 5′ hairpin-forming nucleic acid sequence located 5′ to a 5′ AAV inverted terminal repeat (ITR) of the rAAV vector; (ii) a 3′ hairpin-forming nucleic acid sequence located 3′ to a 3′ AAV inverted terminal repeat (ITR) of the rAAV vector; or (iii) a 5′ hairpin-forming nucleic acid sequence located 5′ to a 5′ AAV inverted terminal repeat (ITR) and a 3′ hairpin-forming nucleic acid sequence located 3′ to a 3′ AAV inverted terminal repeat (ITR) of the rAAV vector.

As used herein, the term “nucleic acid” refers to polymers of linked nucleotides, such as DNA or RNA. In some embodiments, proteins and nucleic acids of the disclosure are isolated. In some embodiments, the DNA of a transgene is transcribed into a messenger RNA (mRNA) transcript. As used herein, the term “isolated” means artificially produced (e.g., an artificially produced nucleic acid, or an artificially produced protein, such as a capsid protein). As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been artificially produced (e.g., by chemical synthesis, by recombinant DNA technology, etc.)

As used herein, a “transgene” is a nucleic acid sequence, which is not homologous to vector sequences, which encodes a polypeptide, protein, functional RNA molecule (e.g., miRNA, miRNA inhibitor) or other gene product, of interest. In some embodiments, a transgene encodes a therapeutic protein or therapeutic functional RNA. Examples of therapeutic proteins include toxins, enzymes (e.g., kinases, phosphorylases, proteases, acetylases, deacetylases, methylases, demethylases, etc.) growth factors, interleukins, interferons, anti-apoptosis factors, cytokines, anti-diabetic factors, anti-apoptosis agents, coagulation factors, anti-tumor factors, and anti-proliferative proteins. The nucleic acid coding sequence is operatively linked to regulatory components in a manner which permits transgene transcription, translation, and/or expression in a cell of a target tissue.

A hairpin-forming nucleic acid (e.g., hairpin-forming DNA), as used herein, refers to a nucleic acid that forms a hairpin structure when two complementary sequences in a single nucleic acid molecule meet and bind together (e.g., form intra-molecular bonds, for example intra-molecular Watson-Crick base pairing). In some embodiments, the hairpin-forming nucleic acid sequence is a hairpin-forming DNA or a hairpin-forming RNA. In some embodiments, the hairpin-forming nucleic acid sequence is a hairpin-forming DNA. In some embodiments, the hairpin-forming DNA encodes a short hairpin RNA (shRNA), a microRNA (miRNA), or an artificial miRNA (amiRNA). In some embodiments, the isolated nucleic acid comprises one hairpin-forming nucleic acid sequence. In some embodiments, the hairpin-forming nucleic acid sequence (e.g., hairpin-forming DNA) is located 5′ to the 5′ ITR of the rAAV vector. In some embodiments, the hairpin-forming nucleic acid (e.g., hairpin-forming DNA) is located 3′ to the 3′ ITR of the rAAV vector. In some embodiments, the isolated nucleic acid comprises two hairpin-forming nucleic acid (e.g., hairpin-forming DNA) sequences flanking the rAAV vector. In some embodiments, the isolated nucleic acid comprises a 5′ hairpin-forming nucleic acid sequence located 5′ to the 5′ AAV inverted terminal repeat (ITR) and a 3′ hairpin-forming nucleic acid sequence located 3′ to the 3′ AAV inverted terminal repeat (ITR).

In some embodiments, additional functional nucleic sequences can be incorporated into a hairpin-forming nucleic acid sequence. In some embodiments, the hairpin-forming nucleic acid comprises a sequence capable of reducing the innate immune response of a subject (e.g., an innate immune response triggered in the subject by administration of an rAAV or other viral vector). In some embodiments, the nucleic acid sequence capable of reducing innate immune response is a TLR9-inhibitory sequence. A TLR9-inhibitory sequence, as used herein, refers to nucleic acid sequences capable of binding to TLR9 and inhibiting TLR9 signaling, thereby inhibiting the TLR9 mediated innate immune response. Examples of TLR9-inhibitory sequence have been previously described, see, e.g., Ashman et al., Optimal oligonucleotide sequences for TLR9 inhibitory activity in human cells: lack of correlation with TLR9 binding, Int Immunol. 2011 March; 23 (3): 203-214; and U.S. Pat. No. 10,190,122, the entire contents of which are incorporated by reference herein. In some embodiments, the hairpin-forming nucleic acid sequence comprises a nucleic acid sequence at least 60%, at least 70%, at least 80%, at least 85%, at least 90%, at least 91%, at least 92%, at least 93%, at least 94%, at least 95%, at least 96%, at least 97%, at least 98%, at least 99%, 100% identical to 5′-TTAGGGTTAGGGTTAGGGTTAGGGTTCAAGAGAccctaaccctaaccctaaccctaa-3′ (SEQ ID NO: 1).

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene.

The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product (e.g., a therapeutic protein or therapeutic minigene) or inhibitory RNA (e.g., shRNA, miRNA, amiRNA, miRNA inhibitor) from a transcribed gene.

The isolated nucleic acids of the disclosure may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise, as disclosed elsewhere herein, one or more regions that encode one or more proteins and/or one or more binding sites for inhibitory nucleic acids (e.g., shRNA, miRNAs, etc.). The transgene may also comprise a region encoding, for example, a protein and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). The AAV ITR sequences may be obtained from any known AAV, including presently identified mammalian AAV types. In some embodiments, the isolated nucleic acid (e.g., the rAAV vector) comprises at least one ITR having a serotype selected from AAV1, AAV2, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125, and variants thereof. In some embodiments, the isolated nucleic acid comprises a region (e.g., a first region) encoding an AAV2 ITR. In some embodiments, the isolated nucleic acid comprises two regions (e.g., a first region and a second region) encoding a 5′ AAV2 ITR and a 3′ AAV2 ITR.

In some embodiments, the isolated nucleic acid further comprises one or more AAV ITRs. In some embodiments, an AAV ITR has a serotype selected from AAV1, AAV2, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125, and variants thereof. In some embodiments, an AAV ITR is a mutant ITR (mTR) that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ATRS ITR). Without wishing to be bound by any particular theory, a rAAV vector comprising an ITR lacking a functional TRS produces a self-complementary rAAV vector, for example as described by McCarthy (2008) Molecular Therapy 16(10):1648-1656.

In some embodiments, the AAV vector described herein is a single-stranded AAV (ss-AAV) vector. As used herein, the term “single-stranded AAV vector” refers to a vector that the coding sequence and complementary sequence of the transgene are on separate strands and are packaged in separate viral capsids. In some embodiments, the AAV vector described herein is a self-complementary (sc-AAV). As used herein, the term “self-complementary AAV vector” (scAAV) refers to a vector that both the coding and complementary sequence of the transgene expression cassette are present on each plus-and minus-strand genome. In some embodiments, a scAAV vector contains a double-stranded vector genome generated by the absence of a terminal resolution site (TR) from one of the ITRs of the AAV. The absence of a TR prevents the initiation of replication at the vector terminus where the TR is not present. In general, scAAV vectors generate single-stranded, inverted repeat genomes, with a wild-type (wt) AAV TR at each end and a mutated TR (mTR) in the middle. In some embodiments, isolated nucleic acids comprise DNA sequences encoding RNA hairpin structures (e.g. shRNA, miRNA, and amiRNA) that can serve a function similar to a mutant inverted terminal repeat (mTR) during viral genome replication, generating self-complementary AAV vector (scAAV) genomes. For example, in some embodiments, the disclosure provides rAAV (e.g. self-complementary AAV; scAAV) vectors comprising a single-stranded self-complementary nucleic acid with inverted terminal repeats (ITRs) at each of two ends and a central portion comprising a promoter operably linked with a sequence encoding a hairpin-forming RNA (e.g., shRNA, miRNA, amiRNA, etc.). In some embodiments, the sequence encoding a hairpin-forming RNA (e.g., shRNA, miRNA, ami-RNA, etc.) is substituted at a position of the self-complementary nucleic acid normally occupied by a mutant ITR.

“Recombinant AAV (rAAV) vectors” are typically composed of, at a minimum, a transgene and its regulatory sequences, and 5′ and 3′ AAV inverted terminal repeats (ITRs). It is this recombinant AAV vector which is packaged into a capsid protein and delivered to a selected target cell. In some embodiments, the transgene is a nucleic acid sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue.

### Recombinant Adeno-Associated Virus (rAAV) and Methods for Producing the Same

In some aspects, the disclosure provides recombinant adeno-associated virus (rAAV) comprising the isolated nucleic acid described herein and an rAAV capsid protein.

Recombinant adeno-associated virus (rAAV) particles are produced by introducing into a host cell, a nucleic acid comprising a transgene, a helper nucleic acid encoding adenoviral helper genes, and a packaging nucleic acid encoding Rep and/or Cap genes. A nucleic acid comprising a transgene may comprise a transgene flanked by adeno-associated virus (AAV) inverted terminal repeats (ITRs). In some embodiments, a helper nucleic acid encoding adenoviral helper genes comprises genes that mediate AAV replication (e.g., AAV E4, E2a and/or VA genes). In some embodiments, a packaging nucleic acid encodes one or more Rep genes. In some embodiments, a packaging nucleic acid encodes one or more Cap genes. As described herein, the methods of producing rAAV particles involve culturing a host cell which contains a nucleic acid sequence encoding an AAV capsid protein; a functional rep gene; a recombinant AAV vector composed of, AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner. In some embodiments, an AAV capsid protein is of an AAV serotype selected from the group consisting of AAV1, AAV2, AAV3, AAV4, AAV5, AAV6, AAV7, AAV8, AAV9, AAV9.hr, AAVrh8, AAVrh10, AAVrh39, AAVrh43, AAV.PHP, AAV.rh32.33 and variants of any of the foregoing.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain El helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. Virol., 70:520-532 (1993) and U.S. Pat. No. 5,478,745.

In some embodiments, the present disclosure methods of reducing reverse packaging of a recombinant adeno-associated virus (rAAV) during rAAV production, the method comprising delivering to host cells: (i) the isolated nucleic acid comprising a recombinant adeno-associated virus (rAAV) vector and (a) a 5′ hairpin-forming nucleic acid sequence located 5′ to the 5′ AAV inverted terminal repeat (ITR); (b) a 3′ hairpin-forming nucleic acid sequence located 3′ to the 3′ AAV inverted terminal repeat (ITR); or (c) a 5′ hairpin-forming nucleic acid sequence located 5′ to the 5′ AAV inverted terminal repeat (ITR) and a 3′ hairpin-forming nucleic acid sequence located 3′ to the 3′ AAV inverted terminal repeat (ITR); (ii) a first vector encoding one or more helper gene; and (iii) a second vector encoding a AAV replication gene and a AAV capsid gene.

Not wishing to be bound to a particular theory, in some embodiments, the presence of the hairpin-forming nucleic acid sequence in the isolated nucleic acid described herein alleviate the package of non-vector DNA (e.g., reverse packaging) by suppressing the read-through genome replication or a reversed packaging. In some embodiments, the presence of the hairpin-forming nucleic acid sequence in the isolated nucleic acid described herein reduce reverse-packaged rAAV particles by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 95%, or 100%.

### Host Cells

In some embodiments, a rAAV production system as described by the disclosure further comprises a host cell. A “host cell” refers to any cell that harbors, or is capable of harboring, a substance of interest. In some embodiments, a host cell is a eukaryotic cell. In some embodiments, a host cell is a mammalian cell. In some embodiments, a mammalian cell is a HEK293 cell, a HEK293T cell, a HeLa cell, a A549 cell, or a Chinese hamster ovary (CHO) cell. In some embodiments, a host cell is a bacterial cell, for example an E. coli cell.

A host cell may be used as a recipient of an isolated nucleic acid or vector as described herein, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants. As used herein, the term “stable cell line” refers to a genome in which the information content of the genome from one generation to the next is maintained.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

The disclosure is based, in part, on isolated nucleic acids and vectors that comprise components needed to replicate and package recombinant adeno-associated virus particles. In some embodiments, an isolated nucleic acid or vector as described herein lacks one or more genes required for replication and/or packaging of rAAV. In some embodiments, an isolated nucleic acid or vector as described herein lacks Ad E1a helper element. In some embodiments, a host cell expresses (or is capable of expressing) the one or more helper elements missing from the isolated nucleic acid or vector. For example, in some embodiments, a host cell expresses Adenovirus helper element Ad-E1a. In some embodiments the Ad-E1a is integrated into the genome of the host cell (e.g., HEK293 cells). In some embodiments, the Ad-E1a is introduced into a host cell and/or transiently expressed in a host cell (e.g., CHO cells).

### rAAV Production Methods

In some aspects, the disclosure provides methods for producing a recombinant adeno-associated virus (rAAV), comprising the step of introducing an rAAV production system as described by the disclosure into a host cell that expresses an Ad-E1a helper function.

Generally, methods described by the disclosure involve transfecting a population of host cells (e.g., host cells expressing Ad-E1a) one or more vectors. In some embodiments, the cap gene and the mutated rep gene as provided by this disclosure are present in the host cell, for example, they are stably integrated in the host cell genome. In some embodiments, the mutated rep gene as described by this disclosure but not the cap gene are present in the host cell, for example, being stably integrated in the host cell genome. In some embodiments, orthogonal aminoacyl-tRNA synthetase-tRNA pairs are present in the host cell genome, to which ncAAs can be supplied. In some embodiments, the one or more vectors comprise Adenoviral helper elements (e.g., Ad5-VA, Ad5-E2a, Ad5-E2b, or Ad5-E4), and/or anticodon engineered synthetic suppressor tRNAs, and/or rAAV cap genes. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells. In some embodiments, the more than one vector of an rAAV production system are introduced into the host cell in a single transfection reaction. In some embodiments, a first vector and second vector of an rAAV production system are introduced into the host cell in separate transfection reactions.

After transfection with the isolated nucleic acids and/or vectors described herein, the host cells can be cultured in the presence of an antibiotic agent that is cognate to the antibiotic-resistance gene of the first vector (e.g., the first vector of the rAAV production system). For example, in some embodiments, a vector comprises an kanR gene and the transfected host cells are cultured in the presence of kanamycin. The concentration of antibiotic agent present in the culture media can vary. In some embodiments, the concentration of antibiotic agent in the culture media ranges from about 5-100 μg/mL (e.g., any amount between 5 and 100 μg/mL, inclusive).

In some embodiments, methods described by the disclosure further comprise the step of supplementing the cell culture after transfection with cognate non-canonical amino acid (ncAA) for the expression of a functional rep gene. In some embodiments, methods described by the disclosure further comprise the step of supplementing the cell culture after transfection with a read-through small molecule for the expression of a functional rep gene.

In some embodiments, methods described by the disclosure further comprise the step of isolating rAAV particles (e.g., rAAV particles comprising the transgene) from the host cells and/or cell culture media. Methods of rAAV purification are known in the art and are described, for example by WO2010148143, WO2016/114992, Potter et al. Mol Ther Methods Clin Dev. 2014; 1:14034, and Wang et al. Methods Mol Biol. 2011;807:361-404.

The disclosure relates, in part, to cell culture systems comprising rAAV production systems described herein. In some aspects, the disclosure provides an apparatus for production of recombinant adeno-associated virus (rAAV) particles, the apparatus comprising: a container housing an rAAV production system as described herein; and, a population of host cells, wherein the rAAV production system and the host cells are suspended in a cell culture medium.

In some embodiments, the container is a cell culture flask, cell culture plate, a beaker, or a cell culture bag. In some embodiments, the cell culture medium is a mammalian cell culture medium. Examples of cell culture media are described, for example, by Yao et al. (2017) Reproductive Medicine and Biology 16(2):99-117.

The disclosure is based, in part, on the recognition that transformation of host cells with isolated nucleic acids and vectors (e.g., rAAV production systems) described by the disclosure allow for production of rAAV viral particles that is cost and time-efficient relative to currently available rAAV production methods (e.g., the triple-transfection method). Methods of measuring viral titer (and/or viral genome copy number) are known in the art and include, for example, silver-stain gel analysis, digital droplet (dd) polymerase chain reaction (ddPCR), and microscopic image analysis. In some embodiments, methods as described by the disclosure produce a viral titer of less than 10¹⁶ rAAV particles (e.g., 10¹⁵, 10¹⁴, 10¹³, 10¹², 10¹¹, 10¹⁰, etc.). In some embodiments, a titer between 10¹⁰ and 10¹⁶ (e.g., 10¹⁵, 10¹⁴, 10¹³, 10¹², 10¹¹, 10¹⁰, or any integer therebetween) rAAV particles are produced.

### EXAMPLES

It has been observed that secondary structure of nucleotide sequences forming palindromic/hairpin shapes hinder AAV genome replication by redirecting the genome replication machinery during AAV vector manufacturing. This example describes placement of short hairpin-encoding sequences outside of AAV inverted terminal repeats (ITRs) as “stoppers”, in order to alleviate the packaging of non-vector DNA by suppressing read-through genome replication or reversed packaging. In order to reduce packaging prokaryotic DNA sequences, the rAAV vectors were engineered to include TLR9-inhibitory sequences into the short hairpin DNA (shDNA). This may also reduce the innate immune response of a cell or subject to bacteria-derived nucleotides or nucleic acid sequences.

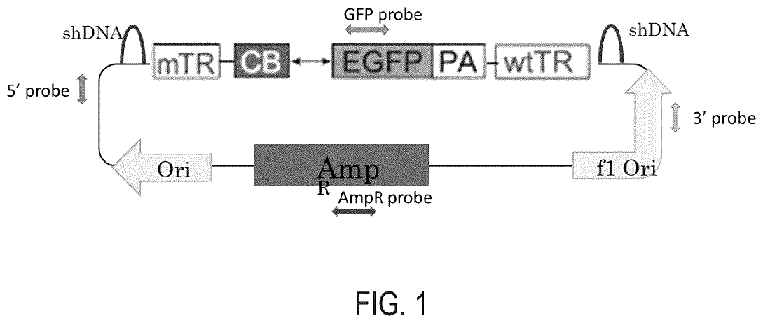

By incorporating shDNA outside of either 5′ or 3′-ITR, or both, improvements of on-target genome packaging were observed in both AAV9 and AAVrh32.33 vectors. FIG. 1 shows a schematic depicting one embodiment of rAAV vectors comprising shDNA “stoppers” positioned outside of the rAAV vector 5′ and 3′ ITRs. The shDNA sequence used was 5′-TTAGGGTTAGGGTTAGGGTTAGGGTTCAAGAGAccctaaccctaaccctaaccctaa-3′ (SEQ ID NO: 1).

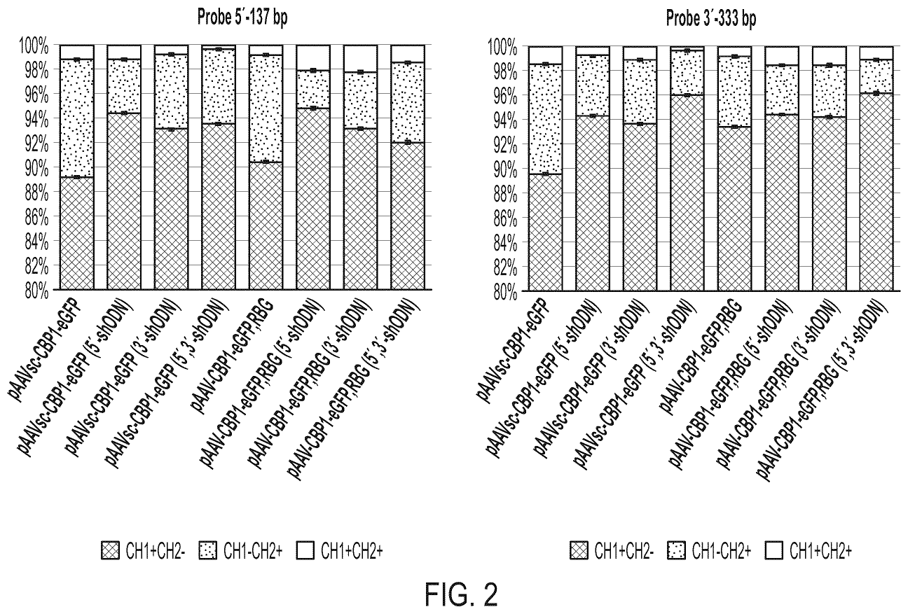

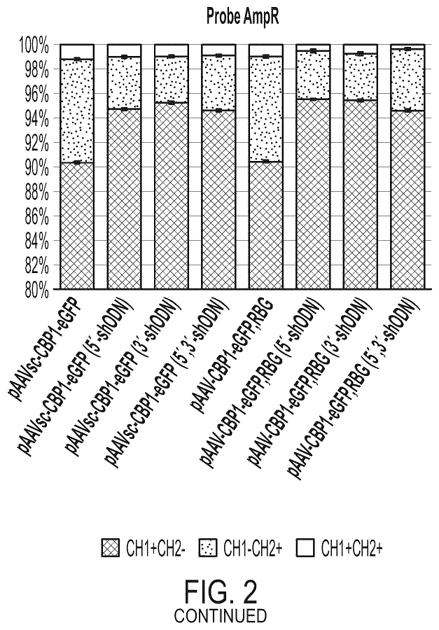

Plasmids for deltaF6, AAV9 capsid, and “Stopper (shDNA)” linked-ITRs encoding a eGFP transgene were exogenously expressed in HEK 293 cells. After 72 hours, crude lysates were harvested for absolute quantification of ‘ROI (region of interest)’ by designing nucleotide specific primer-probes sets of ddPCR. FIG. 2 shows representative data indicating that inclusion of the shDNA “stoppers” on the outside of each of the rAAV ITRs reduced packaging of non-vector DNA into rAAV particles.

shDNAs were incorporated outside of the ITRs in self-complementary (scAAV) or single-stranded AAV (ssAAV) vector plasmids. The scAAV vector was packaged with AAV.rh32.33 capsid protein, while the ssAAV vector was packaged with AAV8 capsid protein. Vector purification was performed by two rounds of CsCl gradient ultracentrifugation or by iodixanol gradient, respectively. Digital droplet PCR analysis of the scAAV.rh32.33 vectors showed that the stopper sequence reduced reverse packaging by more than two-fold (from 10.2% reverse-packaged genomes without stopper, to 4.3% with stopper). The vector genomes extracted from purified scAAV.rh32.33 and ssAAV8 vectors were analyzed by AAVGP-Seq. This sequencing analyses confirmed that the stopper sequences indeed reduced reversed packaged genomes in both scAAV.rh32.33 and ssAAV8 vectors by two-fold. In summary, incorporation of shDNAs beyond the ITRs can reduce the frequency of reverse packaging in both sc-and ssAAV vectors independent of vector purification methods and serotypes.

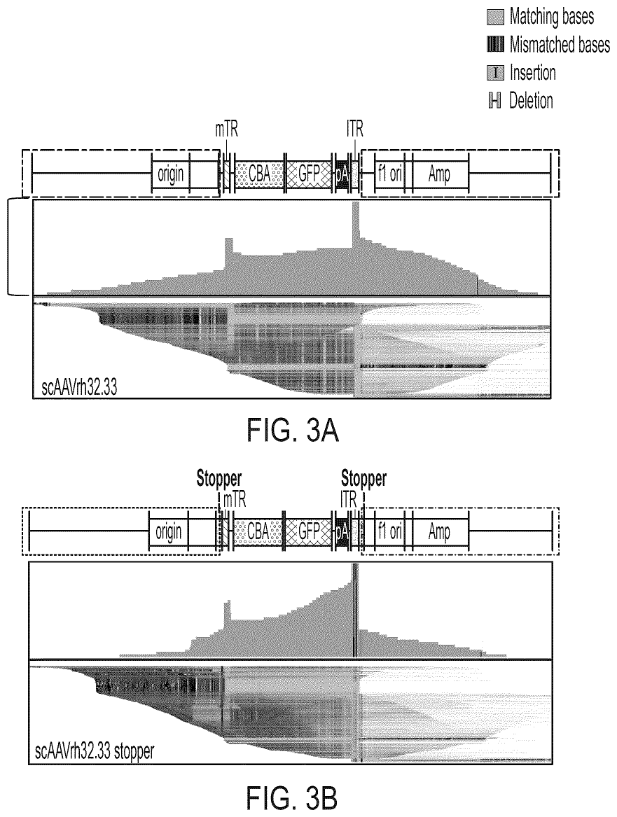

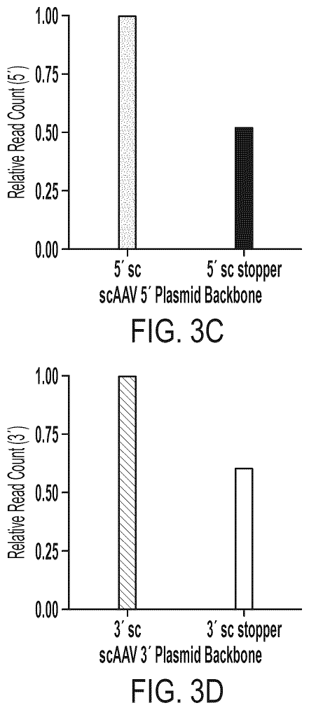

The efficacy of utilizing stopper shDNA in the packaging of vector genomes was further measured using single-molecule real-time (SMRT) sequencing. FIGS. 3A-3B depict schematics of the composition of the vectors used to generate self-complementary AA Vs: scAA Vrh32.33 (FIG. 3A), and scAAVrh32.33 stopper (FIG. 3B). Both vectors contained coding regions for GFP under the control of a CBA promoter, but while the base scAAVrh32.33 vector did not contain shDNA, the scAAVrh32.33 stopper vector contained stopper sequences incorporated outside of both the 5′ and 3′ ITRs. Shown below the vector schematics in FIGS. 3A-3B are visual representations of the SMRT sequencing results corresponding to the different regions of the vectors. The amount of non-vector genome packaged in the AAVs produced from either type of vector was quantified in both the 5′ and 3′ regions of the AAV genomes. FIGS. 3C-3D show graphs of the amount of reverse packaging detected in the 5′ (FIG. 3C) and 3′ (FIG. 3D) regions of the scAAVrh32.33 and scAAVrh32.33 stopper vectors. Consistent with the ddPCR data shown in FIG. 2, the SMRT sequencing data confirmed that less non-vector DNA was packaged in the presence of stopper sequences. These results indicate that the presence of stoppers in the AAV genome can increase the packaging of vector genome, and decrease non-vector genome packaging by approximately 50%.

Introducing “Stopper (shDNA)” at the outside of ITRs has advantages: 1) it can reduce the incorporation of reverse packaged prokaryotic DNAs without affecting AAV yields, and 2) if reverse packaging occurs despite the existence of the stopper sequence, the TLR9-inhibitory sequences engineered into the Stopper shDNA reduce the immune responses to reverse packaged prokaryotic nucleotides in the subject. In some embodiments, the rAAV vectors described in this example are advantageous over previously described constructs and methods for reducing non-vector DNA, for example “stuffering” the vector plasmid backbone to make the genome too large to be packaged, or using minicircle plasmids which contain no bacterial sequence, for vector production.

### EQUIVALENTS

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. An isolated nucleic acid comprising a recombinant adeno-associated virus (rAAV) vector, and
(i) a 5′ hairpin-forming nucleic acid sequence located 5′ to a 5′ AAV inverted terminal repeat (ITR) of the rAAV vector;
(ii) a 3′ hairpin-forming nucleic acid sequence located 3′ to a 3′ AAV inverted terminal repeat (ITR) of the rAAV vector; or
(iii) a 5′ hairpin-forming nucleic acid sequence located 5′ to a 5′ AAV inverted terminal repeat (ITR) and a 3′ hairpin-forming nucleic acid sequence located 3′ to a 3′ AAV inverted terminal repeat (ITR) of the rAAV vector.

2. The isolated nucleic acid of claim 1, wherein each hairpin-forming nucleic acid sequence independently encodes a short hairpin RNA (shRNA), a microRNA (miRNA), or an artificial miRNA (amiRNA).

3. The isolated nucleic acid of claim 1 or 2, wherein each of the hairpin-forming nucleic acid sequences ranges from about 5 nucleotides in length to about 150 nucleotides in length.

4. The isolated nucleic acid of any one of claims 1-3, wherein the 5′ hairpin-forming nucleic acid sequence further comprises a toll-like receptor 9 (TLR9)-inhibitory sequence.

5. The isolated nucleic acid of any one of claims 1-4, wherein the 3′ hairpin-forming nucleic acid sequence further comprises a toll-like receptor 9 (TLR9)-inhibitory sequence.

6. The isolated nucleic acid of any one of claims 1-5, wherein each of the 5′ hairpin-forming nucleic acid sequence and the 3′ hairpin-forming nucleic acid further comprises a toll-like receptor 9 (TLR9)-inhibitory sequence.

7. The isolated nucleic acid of any one of claims 1-6, wherein the rAAV vector is a single-stranded rAAV (ssAAV) vector.

8. The isolated nucleic acid of any one of claims 1-6, wherein the rAAV vector is a self-complementary rAAV (scAAV) vector.

9. The isolated nucleic acid of any one of claims 1-8, wherein the 5′ ITR and/or 3′ ITR is an AAV1, AAV2, AAV3, AAV4, AAV5, AAV6, AAV7, AAV8, or AAV9 ITR.

10. The isolated nucleic acid of any one of claims 1-9, wherein the rAAV vector further comprises a transgene.

11. A recombinant adeno-associated (rAAV) virus comprising:
(i) the isolated nucleic acid of any one of claims 1-10; and
(ii) an adeno-associated virus (AAV) capsid protein.

12. The rAAV of claim 11, wherein the AAV capsid protein is of a serotype selected from AAV1, AAV2, AAV3, AAV4, AAV5, AAV6, AAV7, AAV8, AAV9, AAV.rh32.33, and a variant of any of the foregoing.

13. A cell comprising the isolated nucleic acid of any one of claims 1-10, or the rAAV of claim 11 or 12.

14. A pharmaceutical composition comprising the isolated nucleic acid of any one of claims 1-10, the rAAV of claim 11 or 12, or the cell of claim 13.

15. The pharmaceutical composition of claim 14, further comprising a pharmaceutically acceptable carrier.

16. A method of reducing reverse packaging of a recombinant adeno-associated virus (rAAV) during rAAV production, the method comprising delivering to a host cell:
(i) the isolated nucleic acid of any one of claims 1-10;
(ii) a first vector encoding one or more helper gene; and
(iii) a second vector encoding an AAV replication gene and an AAV capsid gene.

17. A method of reducing reverse packaging of a recombinant adeno-associated virus (rAAV) during rAAV production, the method comprising delivering to a host cell:
(i) an isolated nucleic acid comprising the recombinant adeno-associated virus (rAAV) vector, and
(a) a 5′ hairpin-forming nucleic acid sequence located 5′ to a 5′ AAV inverted terminal repeat (ITR);
(b) a 3′ hairpin-forming nucleic acid sequence located 3′ to a 3′ AAV inverted terminal repeat (ITR); or
(c) a 5′ hairpin-forming nucleic acid sequence located 5′ to a 5′ AAV inverted terminal repeat (ITR) and a 3′ hairpin-forming nucleic acid sequence located 3′ to a 3′ AAV inverted terminal repeat (ITR);
(ii) a first vector encoding one or more helper gene; and
(iii) a second vector encoding an AAV replication gene and an AAV capsid gene.

18. The method of claim 16 or 17, wherein the host cells are human cells or insect cells.

19. The method of claim 18, wherein the human cells are HEK293 cells or HeLa cells.

20. A recombinant adeno-associated virus (rAAV) produced by the method of any one of claims 16 to 19.


SEQUENCE LISTING (1 sequence)

SEQ ID NO: 1 - DNA, 57 bp, synthetic construct
  source 1..57: mol_type = other DNA
ttagggttagggttagggttagggttcaagagaccctaaccctaaccctaaccctaa